(19) United States
(12) Patent Application Publication
(10) Pub. No.: US 2008/0287932 A1
(43) Pub. Date: Nov. 20, 2008

(54) Apparatus and Method for Treatment and Particularly Laser Treatment of a Cancer or Precancerous Condition

(76) Inventors: Jaouad Zemmouri, Hem (FR); Igor Razdobreev, Faches-Thumesnil (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/586,082

(22) PCT Filed: Jan. 10, 2005
(86) PCT No.: PCT/EP05/00128

§ 371 Date: Jul. 30, 2008

(30) Foreign Application Priority Data
Jan. 14, 2004 (FR) 0400283

(51) Int. Cl. A61N 5/067

(52) U.S. Cl. 606/10

(57) ABSTRACT

An apparatus for treatment of a cancer or precancerous condition which includes a therapeutic light source for outputting a therapeutic light beam at a wavelength between 1.2 μm and 1.3 μm. This light source is preferably a laser for outputting a pulsed beam.

This invention relates to the field of oncology, and concerns an apparatus and a method for treating cancer or a precancerous state by means of a therapeutic light beam, and in particular a laser beam that is not necessarily thermal.

### PRIOR ART

In general, cancer is a cellular process that involves the appearance, starting with a normal cell, of cells having a generally abnormal morphology and behaviour, called cancer cells. These cancer cells thrive at the expense of normal cells and arrange themselves to form cancerous tumours. Cancer tissue is thus formed by:
actual cancer cells arranged in substantially structured formations and corresponding to the cancerous tumour, and
the stroma, i.e. a connective tissue providing support and nutrition for the cancerous tumour.

The development of cancer passes through various known stages, from the development of the initial clone from a strain cell to the metastatic dissemination. In addition, prior to the appearance of the actual cancer, the subject goes though so-called precancerous states. The World Health Organisation distinguishes two types of precancerous states:
precancerous conditions that are clinical states associated with a significantly high risk of cancer,
precancerous lesions, namely histopathological abnormalities, which, if they persist for a long enough time, can result in the appearance of cancer. These precancerous lesions are also called dysplasias.

To treat cancer tissue or a precancerous lesion, it is currently known in oncology to use a so-called photodynamic therapy method (PDT). This relatively new method is intended to destroy cancer cells by photochemical reactions. This method consists, in a first step, of marking the area to be treated (cancer tissue or precancerous lesion) with a photosensitising product, then, in a second step, of lighting the area to be treated with a laser beam that has an appropriate wavelength preferably absorbed by the photosensitising product, and that makes it possible to activate the photosensitising product and produce cytotoxic compounds ensuring the in situ destruction of the cancer cells. Depending on the type of cancer or precancerous state to be treated, the photosensitising product can be injected intravenously, administered orally or be applied directly at the surface of the area to be treated: for example, the treatment of skin cancers, the treatment of actinic keratoses, which are precancerous lesions of the skin caused by photoaging, and so on.

One of the advantages of photodynamic therapy lies in the possibility of using low-power lasers (“non-thermal” lasers) that cause little or nor thermal effects in the treated area, and, therefore, are not destructive.

However, photodynamic therapy has a number of disadvantages. The first disadvantage is associated with the patient's photosensitisation, which makes it necessary to avoid any sun exposure for a relatively long period, generally around 48 hours. A second disadvantage is associated with the use of an expensive drug (photosensitising product), which makes this treatment costly, especially since the treatment must be repeated several times in order to be effective. A third disadvantage lies in the appearance in some patients of adverse effects associated with the injection or application of the photosensitising product.

### OBJECTIVE OF THE INVENTION

This invention is intended to provide a new solution for the treatment of cancer or a precancerous state, which has the advantages of PDT, in that it uses a “non-thermal” therapeutic light beam, but that does not require the use of a photosensitising product.

### SUMMARY OF THE INVENTION

The invention thus relates to an apparatus for the treatment of cancer or a precancerous state, which, in a manner known per se, comprises a therapeutic light source.

Characteristically according to the invention, the light source is designed to emit a therapeutic light beam having a wavelength between 1.2 μm and 1.3 μm.

The invention also relates to a method for treating cancer or a precancerous state in which the site to be treated is illuminated with a therapeutic light beam having a wavelength between 1.2 μm and 1.3 μm, preferably without the prior administration of a photosensitising drug such as that used in the case of PDT.

The treatment apparatus is preferably more specifically characterised by one and/or the other of the following additional features, alone or in combination with one another:
the source is designed to emit a therapeutic pulsed light beam;
the time of each pulse can be adjusted;
the time of each pulse can be set to a value less than 0.5 s, and preferably at least between 0.1 s and 0.3 s;
the time interval between two pulses is adjustable;
the time interval between two pulses can be set to a value greater than 0.5 s, and
preferably to a value greater than or equal to 0.9 s;
the time of emission of the therapeutic light beam is adjustable;
the number of pulses in each emission is adjustable;
the number of pulses in each emission can be set to at least between 50 and 300;
the power of the therapeutic light beam is adjustable;
the power of the therapeutic light beam can be set to at least between 1 W and 5 W;
the power density of the pulses can be set to at least between 30 W/cm² and 300 W/cm²;
the source is a laser source;
the laser source comprises a Raman fiber laser;
the Raman fiber laser includes a pump laser diode, an ytterbium-doped fiber laser, and a Raman converter that is intended to transpose the wavelength of the beam generated by the ytterbium-doped fiber laser.

The treatment method according to the invention preferably has one and/or the other of the following additional characteristics, alone or in combination with one another:
the therapeutic light beam is advantageously a pulsed beam;
the power density (d) of the laser beam at the level of the site to be treated is preferably between 30 W/cm² and 300 W/cm², and is more preferably on the order of 100 W/cm²;
the pulse fluence is preferably between 1 J/cm² and 30 J/cm²;
the total fluence for each emission is between 6000 J/cm² and 90,000 J/cm², and is more preferably on the order of 30,000 J/cm²;
the time (T) between two successive pulses is greater than 0.5 s, and more specifically greater than or equal to 0.9 s;
the number of pulses (N) in each emission is preferably between 50 and 300 pulses;
the time (t) of each pulse is preferably less than 0.5 s, and more preferably between 0.1 sand 0.3 s;
the operation of lighting the site to be treated is repeated a number of times, preferably with at least one day of rest between each lighting operation.

It has been noted that the use of a therapeutic light beam having the aforementioned wavelength and power characteristics advantageously and surprisingly makes it possible to obtain satisfactory results in the treatment of precancerous states or cancer, without requiring the use of a drug as in the case of PDT. Therefore, it can be assumed that the action of this therapeutic light beam in the aforementioned power and wavelength range would make it possible to generate the singlet oxygen directly from the oxygen contained in the cancer cells, and in a sufficient amount to produce necrosis of the cancer cell, in a manner comparable to that produced with PDT with a drug activated by a light beam. However, the inventors are not bound by this explanation.

### DESCRIPTION OF THE FIGURE

Other characteristics and advantages of the invention become clearer from the following description of a preferred embodiment of a treatment apparatus of the invention and the use thereof, which description is provided by way of a non-limiting example, in reference to the appended

FIG. 1 showing a general diagram of a treatment apparatus according to the invention.

### DETAILED DESCRIPTION

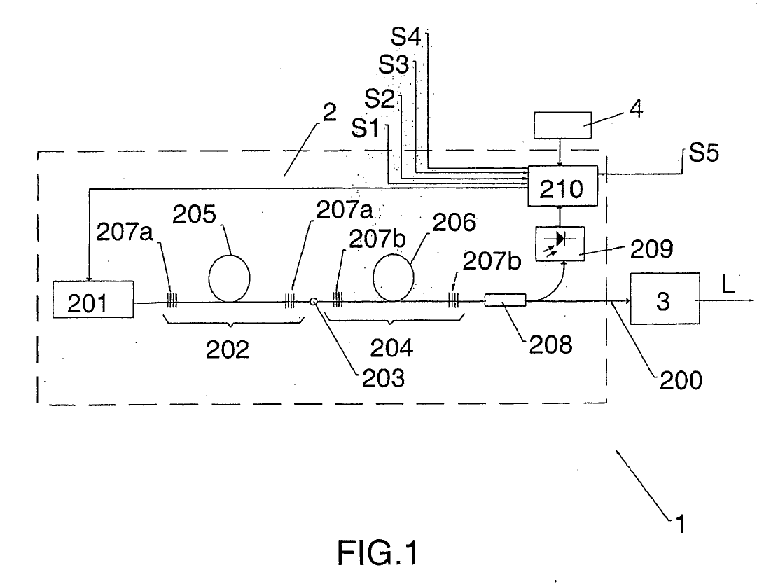

In reference to the diagram of the appended FIG. 1, the apparatus 1 for treating cancer or a cancerous state essentially comprises a light source 2 with a fiber output 200, and an adaptation interface 3. The adaptation interface 3 generally makes it possible to direct the therapeutic light beam (L), generated at the output 200 by the source 2, to the site to be treated.

The adaptation interface 3 is known per se to a person skilled in the art and, therefore, will not be described in detail in this description. It is chosen by a person skilled in the art on the basis of the type of cancer or precancerous state to be treated, in a manner comparable to that performed in the context of PDT. The following are non-limiting and non-exhaustive examples of the invention:
in dermatology or surgery, the adaptation interface 3 is a handpiece that enables the practitioner to bring the beam as close as possible to the cancerous tumour or the precancerous lesion to be treated;
in ORL and opthalmology, the adaptation interface 3 can be a handpiece, a biomicroscope or a slit lamp with a sighting laser,
in gastroenterology, pneumology, urology and gynaecology, the adaptation interface 3 is an endoscope.

According to a first characteristic of the invention, and regardless of the adaptation interface 3, the light source 2 is designed to emit, at the output 200, a therapeutic light beam having an emission wavelength between 1.2 μm and 1.3 μm.

This therapeutic light beam is preferably a coherent light beam (laser). Nevertheless, in another embodiment, the therapeutic light beam can be an incoherent light beam, generated by a light source having a sufficient power followed by optical filtering so as to retain only the frequency components in the range of 1.2 μm to 1.3 μm.

In reference to FIG. 1, the light source 2 of the apparatus 1 also comprises means (208, 209, 210, S1, S2, S3, S4, S5) enabling the practitioner to adjust the main beam (L) emission parameters (in particular, power, number of pulses, time of each pulse, time interval between two pulses); these adjustment means will be described below in greater detail.

The apparatus 1 also comprises control means 4, which enable the practitioner to control the activation of the therapeutic light beam according to the emission parameters that have been set. These control means 4 comprise, for example, an action pedal or any other equivalent manual activation means.

When the therapeutic light beam is a laser beam, in its most general sense, the invention is not limited to a specific type of laser source 2, as any laser source allowing for the emission of a laser beam satisfying the aforementioned wavelength condition, and known to a person skilled in the art, can be used. In particular, in a non-exhaustive manner, it is possible to use the following types of laser source:
Raman fiber laser, continuous or pulsed;
Cr: Forsterite (Cr₄₊: Mg₂SIO₄) laser, pulsed or continuous, pumped by a neodymium (Nd)-doped solid or fiber laser, by an ytterbium-doped solid or fiber laser, or diode-pumped;
pulsed or continuous parametric oscillator, pumped by another laser source;
power laser diode;
solid continuous or pulsed Raman converter or laser pumped by another laser source.

Among the lasers mentioned above, a Raman fiber laser is preferably used for the following reasons:
the fiber output of the laser facilitates the transport of the beam to the output 200;
the laser beam generated has a good spectral and spatial quality;
the laser source 2 is advantageously compact;
the laser source 2 is reliable and does not require any maintenance;
this type of laser source provides the best compromise between quality and production cost of the laser.
   Preferred Embodiment of a Raman Fiber Laser with a Wavelength Between 1.2 μm and 1.3 μm

In reference to FIG. 1, the source 2 is a Raman fiber laser and comprises a pump laser diode 201 with a wavelength of 910-930 nm or 970-980 nm, an ytterbium (Yb)-doped fiber laser 202, and a Raman converter 204 that is intended to transpose the wavelength of the beam at the output of the fiber laser 202, so as to obtain a laser beam with a wavelength of 1260-1270 nm.

The ytterbium (Yb)-doped fiber laser 202 consists of a double-coated fiber 205 of which the core is doped with ytterbium and two Bragg gratings 207a at the input and output, which are photoinscribed in the fiber. The output 203 of the fiber of the laser 202 is directly welded to the input of the Raman converter 204.

The Raman converter 204 includes a fiber 206 of which the core is doped with phosphorus and two Bragg gratings 207b at the input and the output, which are set to a wavelength in the range of 1260-1270 nm. This converter 204 makes it possible to perform the transposition of the emission wavelength of the laser 202 in a single step.

In another alternative, it is possible to use a monomode fiber, different from the aforementioned fiber; it is appropriate in this case to adapt the number of steps in the conversion of the Raman 204 converter according to the nature of the fiber, and in particular the type of doping agent used.

It is also possible to replace the Bragg gratings with monomode couplers.

The Raman fiber laser described above in reference to FIG. 1, which allows for the emission of a therapeutic laser beam at a wavelength between 1.2 μm and 1.3 μm, is novel per se, and can therefore advantageously be used in other applications (medical or non-medical), outside of the specific field of the treatment of cancer or precancerous states.

In reference to FIG. 1, the power of the laser beam is adjusted via a coupler 208 having a low lock-in rate, and a photodiode 209 connected to electronic control means 210. The electronic control means 210 also receive, at the input, a first continuous set point signal (S1) of which the value is manually set by the practitioner (for example, by means of a potentiometer or the like) and that characterises the set point power in continuous mode of the laser beam. From this set point value (signal S1), the electronic control means 210 automatically set the power of the laser beam emitted by acting at the output directly on the current of the pump diode 201. The electronic control means 210 thus enable the practitioner to manually set the power of the therapeutic laser beam at a predefined value (set point signal S1).

In addition, the electronic control means 210 receive, at the input, four other continuous set point signals S2, S3, S4 and S5 of which the values are manually set by the practitioner:
the set point signal S2 characterises, for example, the operation mode (continuous or pulsed);
the set point signal S3 characterises, for example, in the case of a pulsed mode, the time of each pulse of the therapeutic laser beam;
the set point signal S4 characterises, for example, in the case of a pulsed mode, time interval between two successive pulses,
the set point signal S5 characterises the time of emission (or in other words the number of pulses in the case of a pulsed mode) of the therapeutic laser beam, upon each actuation of the control means 4.

The electronic control means 210 thus control the current of the pump diode 201 on the basis of the set point signals S1 to S5 and the signal extracted by the coupler 208 and the photodiode 209, so as to automatically set the physical characteristics of the emitted laser beam (power, mode (pulsed or continuous), emission time, and in the case of a pulsed mode: time of each pulse and time interval between each pulse).

### Treatment Method

The apparatus of the invention is implemented as follows:

Step 1: the practitioner manually sets the emission parameters of the therapeutic laser beam (power, mode (pulsed or continuous), emission time (or number of pulses in the case of a pulsed mode), and in the case of a pulsed mode: time of each pulse and time interval between two pulses).

Step 2: by means of the adaptation interface 3, the practitioner adjusts, in a manner that is very precise and known per se, the spatial position of the laser beam with respect to the cancer or precancerous site to be treated.

Step 3: When the alignment is perfect, the practitioner actuates the control pedal 4, which activates the emission of the therapeutic beam (lighting of the site to be treated) with the predefined emission parameters.

When the targeted site is treated, the practitioner repeats the operations of steps 2 and 3 on another site to be treated, as many times as is necessary to cover the entire surface of the tumour or cancerous or precancerous lesions.

The aforementioned operations are repeated at a frequency according to the treatment protocol determined on a case-by-case basis by the practitioner.

The treatment method of the invention can be used for the treatment of malignant or benign tumours, the treatment of precancerous states, and the post-operative, post-radiation and/or post-chemotherapy treatment of tumours. The treatment can be performed as a complement to surgery, chemotherapy or radiation.

The treatment apparatus can be used to treat all precancerous lesions (dysplasias, carcinomas in situ) or cancers that are accessible by a light beam; it is simply necessary to choose the appropriate adaptation interface according to the location of the site. In particular, the treatment apparatus can be used to treat all precancerous lesions (dysplasias, carcinomas in situ) or cancers that are currently treated by means of PDT. As a non-limiting and non-exhaustive example, the various cancers that can be treated include:
in ORL: cancer of the oral cavity, thyroid cancer, laryngopharynx cancer, larynx cancer, nasopharynx cancer;
digestive system: oesophageal cancer, Barrett's oesophagus, stomach cancer, colon and rectum cancer, pancreatic cancer, gallbladder cancer;
respiratory system: all known types of cancer of the respiratory pathways or the lungs;
in urology: kidney cancer, testicular cancer, bladder cancer, prostate cancer, penile cancer;
in gynecology: cervical cancer, endometrial (uterine) cancer, vaginal cancer;
in dermatology: actinic keratoses, melanomas, basal cell carcinomas, intraepithelial neoplasias and epidermoid carcinomas.

Regardless of the type of cancer or precancerous lesion, it is preferably to use a pulsed laser beam (L), rather than a continuous laser beam, because this makes it possible to reduce the risk of burning tissue.

More specifically, regardless of the type of cancer or precancerous lesion, the treatment method and treatment apparatus of the invention preferably have one and/or the other of the technical features below.

The power density (d) of the laser beam at the level of the site to be treated is preferably between 30 W/cm² and 300 W/cm², and is more preferably on the order of 100 W/cm², with the reminder that the power density (d) is defined by the following formula:

d=P/S

With P representing the pulse power and S representing the surface of the spot formed by the laser beam at the level of the site to be treated.

The pulse fluence is preferably between 1 J/cm² and 30 J/cm². It is noted here that the pulse fluence (F) is defined by the following formula:

F=d×t

in which formula d represents the pulse power density and t represents the pulse duration.

The surface (S) of the spot is dependent on the diameter of the laser beam at the output of the fiber, the “waist” of the beam and the distance between the fiber output of the laser and the site to be treated. For a given waist and diameter of the laser beam, the farther the fiber output of the laser is, the greater the surface of the spot will be, and the lower the power density and the pulse fluence will be.

The total fluence for each emission is preferably between 6000 and 90,000 J/cm², and is more preferably on the order of 30,000 J/cm², with the reminder that the total fluence (FT) for each emission is defined by the following formula:

FT=F×N

where N represents the number of pulses in each emission and F represents the pulse fluence.

The time (T) between two successive pulses must be great enough to prevent any overheating of the tissue. The time (T) between two successive pulses is preferably greater than 0.5 s, and more specifically greater than or equal to 0.9 s.

More specifically, a satisfactory compromise, which makes it possible to comply with the aforementioned fluence values while limiting the treatment time in each emission so as not to immobilise the patient for too long, was obtained with a number of pulses (N) in each emission preferably between 50 and 300 pulses with a time (t) of each pulse between 0.1 s and 0.3 s.

More specifically, the treatment apparatus is preferably characterised by a beam of which the pulse power is between 1 W and 5 W and is more preferably on the order of 3 W, and of which the pulse power density at the output of the apparatus is between 30 W/cm² and 300 W/cm², and more preferably on the order of 100 W/cm².

The treatment protocol is defined by the practitioner in particular according to the size of the tumour or cancerous or precancerous lesion as well as the desired immobilisation time for the patient.

The following is an example of a treatment protocol: daily for a number of days at a time or every three days for a number of days at a time. In every case, it is preferably to repeat the operation of lighting the site to be treated a number of times with at least one day of rest between each lighting operation.

Nevertheless, it should be emphasized that, advantageously, it is possible for the treatment of the invention to cause no harmful adverse effects, and in particular no overheating of the tissue. It is therefore also desirable to shorten the total time of the treatment protocol by performing, in a single day, a number of successive operations of lighting the site to be treated, without being required to provide a day of rest between each operation, as in the aforementioned protocol examples.

However, the invention is not limited to the aforementioned parameters and conditions of use, which are given solely by way of indication.

1. An apparatus for the treatment of cancer or a precancerous state in a patient in need thereof, which apparatus comprises a therapeutic light source, designed to emit a therapeutic light beam having a wavelength between 1.2 μm and 1.3 μm, wherein said therapeutic light beam is sufficient to treat said cancer or precancerous state in said patient.

2. The apparatus according to claim 1, wherein said light source is designed to emit a pulsed therapeutic light beam.

3. The apparatus according to claim 2, wherein the time of each pulse is adjustable.

4. The apparatus according to claim 2, wherein the time of each pulse can be set to a value less than 0.5 s.

5. The apparatus according to claim 2, wherein the time interval between two pulses is adjustable.

6. The apparatus according to claim 5, wherein the time interval between two pulses can be set to a value greater than 0.5 s.

7. The apparatus according to claim 2 wherein the time of emission or the number of pulses in each emission of the therapeutic light beam is adjustable.

8. The apparatus according to claim 2, wherein the number of pulses in each emission can be set to at least between 50 and 300.

9. The apparatus according to claim 1, wherein the power of the therapeutic light beam is adjustable.

10. The apparatus according to claim 9, wherein the power of the therapeutic light beam can be set to at least between 1 W and 5 W.

11. The apparatus according to claims 2 or 9, wherein the power density of the pulses can be set to at least between 30 W/cm² and 300 W/cm².

12. The apparatus according to claim 1, wherein the light source is a laser source.

13. The apparatus according to claim 12, wherein the laser source comprises a Raman fiber laser.

14. The apparatus according to claim 13, wherein the Raman fiber laser includes a pump laser diode (201), an ytterbium-doped fiber laser (202), and a Raman converter (204) that is intended to transpose the wavelength of the beam generated by the ytterbium-doped fiber laser.

15. A method for treating cancer or a precancerous state in a patient in need thereof, comprising illuminating the site to be treated with a therapeutic light beam having a wavelength between 1.2 μm and 1.3 μm, wherein said therapeutic light beam is sufficient to treat said cancer or precancerous state in said patient.

16. The method according to claim 15, wherein the therapeutic light beam is a laser beam.

17. The method according to claim 15, wherein the therapeutic light beam is pulsed.

18. The method according to claim 17, wherein the pulse fluence is between 1 J/cm² and 30 J/cm².

19. The method according to claim 17, wherein the time (T) between two successive pulses is greater than 0.5 s.

20. The method according to claim 17, wherein the time (T) between two successive pulses is greater than or equal to 0.9 S.

21. The method according to claim 17, wherein the number of pulses (N) in each emission is between 50 and 300 pulses.

22. The method according to claim 17, wherein the time (t) of each pulse is less than 0.5 s.

23. The method according to claim 17, wherein the time (t) of each pulse is between 0.1 s and 0.3 s.

24. The method according to claim 15, wherein the power density (d) of the therapeutic light beam at the level of the site to be treated is between 30 W/cm² and 300 W/cm².

25. The method according to claim 15, wherein the power density (d) of the therapeutic light beam at the level of the site to be treated is substantially equal to 100 W/cm².

26. The method according to claim 15, wherein the total fluence for each emission is between 6000 J/cm² and 90,000 J/cm².

27. The method according to claim 15, wherein the total fluence for each emission is substantially equal 30,000 J/cm².

28. The method according to claim 15, wherein the operation of lighting the site to be treated is repeated a number of times, with at least one day of rest between each lighting operation.

29. The method according to claim 15, wherein a photosensitising drug is not administered to the patient.

30. The apparatus according to claim 4, wherein the time of each pulse can be set to a value between at least 0.1 s and 0.3 s.

31. The apparatus according to claim 6, wherein the time interval between two pulses can be set to a value greater than or equal to 0.9 s.
